(19) United States
(12) Patent Application Publication
(10) Pub. No.: US 2024/0148937 A1
(43) Pub. Date: May 9, 2024

(54) ADIPOSE-DERIVED HYDROGEL COMPOSITIONS AND METHODS OF USE

(71) Applicant: University of Massachusetts, Boston, MA (US)

(72) Inventors: Silvia Corvera, Westborough, MA (US); Janice F. Lalikos, Northborough, MA (US); Tiffany DeSouza, Putnam, CT (US)

(21) Appl. No.: 18/283,675

(22) PCT Filed: Mar. 24, 2022
(86) PCT No.: PCT/US22/21693

§ 371 Date: Sep. 22, 2023

Related U.S. Application Data
(60) Provisional application No. 63/165,680, filed on Mar. 24, 2021.

(51) Int. Cl. A61L 27/38; A61L 27/52

(52) U.S. Cl. CPC A61L 27/3804; A61L 27/3834; A61L 27/3839; A61L 27/3895; A61L 27/52

(57) ABSTRACT

Isolated hydrogel compositions derived from adipose tissue and related methods of preparation and use are provided. The isolated hydrogel compositions can include one or more of collagen 1A1, collagen 1A2, collagen 3A1, collagen 4A2, collagen 5A2, and fibrillin-1.

### CLAIM OF PRIORITY

This application claims the benefit of U.S. Provisional Patent Application Ser. No. 63/165,680, filed on Mar. 24, 2021, the entire contents of which are hereby incorporated by reference.

### TECHNICAL FIELD

This disclosure relates to adipose-derived hydrogel compositions and related methods of preparation of these compositions for use in tissue engineering and/or cell therapies.

### BACKGROUND

The success of cell-based therapies in which a patient's own cells are removed, genetically altered, and re-implanted to fight tumors has been one of the most important advances in cancer treatment over the past decade. These successes have underscored the great potential of cell therapies for numerous other therapeutic indications. Pre-clinical work has demonstrated successful use of cell therapies for large medically unmet needs including wound healing, cartilage repair, graft versus host disease, and amyotrophic lateral sclerosis (ALS). Indeed, there are already several examples of cell-based therapies approved by the Food and Drug Administration (FDA) for reconstruction of soft tissue, cartilage, and mucogingival tissue defects. Cell therapies have also been approved in the European Union for treatment of complex perianal fistulas in patients with Crohn's disease.

Beyond these indications, genetic diseases are also amenable to cell therapies. One example is lipodystrophies, in which patients lack adipocytes. Lipodystrophies can range in severity and age of onset, from children lacking all visible fat (congenital generalized lipodystrophy) to young adults that lose fat in their arms and legs over a period of years (familial partial lipodystrophy). Individuals with lipodystrophy develop severe metabolic disease, characterized by hyperlipidemia, type-2 diabetes, hyperinsulinemia, fatty liver, and atherosclerosis.

There is much interest in advancing cell therapies for a wide array of applications ranging from wound healing, genetic diseases, therapies outside the immune-oncology space (e.g., CRISPR-based cell therapy), metabolic diseases. However, a major hurdle for the advancement of cell-based therapies is the paucity of three-dimensional scaffolds, which are required to grow cells in vitro prior to implanting them into the body. More importantly, there is a scarcity of three-dimensional scaffolds that are compatible with Good Manufacturing Practices (GMP) to be used in the manufacturing of cell therapy products. Thus, better three-dimensional scaffolds that are compatible with GMP requirements are necessary.

### SUMMARY

The present disclosure is based, at least in part, on the development of isolated hydrogel compositions derived from adipose tissue and used for applications such as tissue engineering and cell therapies. The methods of preparation of these isolated hydrogel compositions is also described herein.

Thus, in a first aspect, the disclosure features isolated hydrogel compositions including one or more of collagen 1A1, collagen 1A2, collagen 3A1, collagen 4A2, collagen 5A2, and fibrillin-1, wherein the composition is derived from a mammalian, e.g., human, adipose tissue, and wherein the composition is substantially free of nucleic acids and.

In some embodiments, the one or more of collagen 1A1, collagen 1A2, collagen 3A1, collagen 4A2, collagen 5A2, and fibrillin-1 are fragmented.

In some embodiments, the composition comprises one or more peptides of one or more collagen 1A1, collagen 1A2, collagen 3A1, collagen 4A2, collagen 5A2, and fibrillin-1.

In some embodiments, a size of the one or more fragmented collagen 1A1, collagen 1A2, collagen 3A1, collagen 4A2, collagen 5A2, and fibrillin-1 ranges from about 10 kilodalton (kDa) to about 30 kDa.

In some embodiments, collagen 1A1 is present at a concentration greater than a concentration of each of collagen 1A2, collagen 3A1, collagen 4A2, collagen 5A2, and fibrillin-1.

In some embodiments, fibrillin-1 is present at a concentration that is less than a concentration of each of collagen 1A1, collagen 1A2, collagen 3A1, collagen 4A2, and collagen 5A2.

In some embodiments, collagen 1A1 is present at a concentration of about 20 weight (wt) %, collagen 1A2 is present at a concentration of about 18 wt %, collagen 3A1 is present at a concentration of about 17 wt %, collagen 4A2 is present at a concentration of about 16 wt %, collagen 5A2 is present at a concentration of about 15 wt %, and fibrillin-1 is present at a concentration of about 14 wt %.

In some embodiments, collagen 1A1 is present at a concentration ranging from about 15 weight (wt) % to about 25 wt %, collagen 1A2 is present at a concentration ranging from about 13 wt % to about 23 wt % collagen 3A1 is present at a concentration ranging from about 12 wt % to about 22 wt % collagen 4A2 is present at a concentration ranging from about 11 wt % to about 21 wt % collagen 5A2 is present at a concentration ranging from about 10 wt % to about 20 wt % and fibrillin-1 is present at a concentration ranging from about 9 wt % to about 19 wt %.

In some embodiments, the composition is substantially free of cells, nucleic acids, non-fibrous proteins, and/or lipids.

In some embodiments, the composition forms a gel when exposed to a temperature ranging from about 37 degrees Celsius to about 40 degrees Celsius.

In some embodiments, the composition is a liquid when exposed to a temperature ranging from about 1 degree Celsius to about 5 degrees Celsius.

In another aspect, the disclosure provides methods of preparing an isolated hydrogel composition. The methods include providing an adipose tissue sample from a subject; freezing the adipose tissue sample; slicing the adipose tissue sample into a sheet; contacting the sheet with a denaturant to denature substantially all non-fibrous protein in the adipose tissue sample; mechanically processing the adipose tissue sample to lyse substantially all cellular material in the adipose tissue sample; contacting the adipose tissue sample with a nuclease to remove substantially all nucleic acid material from the adipose tissue sample; contacting the adipose tissue sample with an organic solvent to remove substantially all lipids from the adipose tissue sample; contacting the adipose tissue sample with a protease to digest proteins in the adipose tissue sample; and dialyzing the adipose tissue sample using a dialysis membrane.

In some embodiments, the sheet has a thickness ranging from about 1 millimeter (mm) to about 3 mm.

In some embodiments, the method does not comprise contacting the adipose tissue sample with a detergent.

In some embodiments, the denaturant is guanidine hydrochloride.

In some embodiments, the mechanically processing the adipose tissue sample comprises homogenizing the sample.

In some embodiments, the nuclease is an endonuclease.

In some embodiments, the organic solvent is a polar organic solvent and/or a non-polar organic solvent.

In some embodiments, the organic solvent is ethanol, methanol, chloroform, or any combination thereof.

In some embodiments, the protease is pepsin.

In some embodiments, the protease comprises a protease-acid solution having a weight that is about 4 times the weight of the adipose tissue sample.

In some embodiments, the concentration of the protease in the protease-acid solution is about 75%, and the concentration of the acid in the protease-acid solution is about 0.5M.

In some embodiments, the dialysis membrane has a molecular weight cutoff ranging from about 12 kDa to about 14 kDa.

In another aspect, the disclosure provides isolated hydrogel compositions prepared by any of the methods disclosed herein.

In yet another aspect, the disclosure provides methods of culturing a cell or a population of cells. The methods include suspending the cell or the population of cells in or contacting the cell or population of cells with the isolated hydrogel composition of the disclosure, under conditions sufficient for growth of the cell or the population of cells.

In some embodiments, the cell or the population of cells includes one or more of a stem cell, a progenitor cell, a pluripotent cell, an induced pluripotent stem cell (iPSC), and an iPSC-derived beta cell.

In some embodiments, the cell or the population of cells include a cell that has been genetically altered.

In some embodiments, the isolated hydrogel compositions promotes a growth of the cell or the population of cells.

In some embodiments, suspending the cell or the population of cells in the isolated hydrogel composition is performed at a temperature that is at least about 4 degrees Celsius or less.

In some embodiments, the isolated hydrogel composition is in a liquid or flowable state.

In some embodiments, contacting the cell or the population of cells with the isolated hydrogel composition is performed at a temperature ranging from about 6 degrees Celsius to about 40 degrees Celsius.

In some embodiments, contacting the cell or cells with the isolated hydrogel composition is performed at a temperature of about 37 degrees Celsius.

In some embodiments, the isolated hydrogel composition is in a solid or gel state.

In another aspect, the disclosure provides methods of treating a lipodystrophy in a subject in need thereof. The methods can include obtaining a population of adipose progenitor cells from the subject; optionally genetically altering one or more adipose progenitor cells from the population of adipose progenitor cells to correct an underlying genetic defect; optionally differentiating the one or more adipose progenitor cells into adipocytes; suspending the one or more adipose progenitor cells in or contacting the one or more adipose progenitor cells with any of the isolated hydrogel compositions of the disclosure; and implanting the isolated hydrogel composition comprising the one or more adipose progenitor cells into the subject.

In some embodiments, suspending the one or more adipose progenitor cells in the isolated hydrogel composition is performed at a temperature that is at least about 4 degrees Celsius or less.

In some embodiments, the isolated hydrogel composition is in a liquid or flowable state.

In some embodiments, the step of implanting the isolated hydrogel composition comprising the one or more adipose progenitor cells into the subject includes injecting isolated hydrogel composition comprising the one or more adipose progenitor cells in the subject.

In some embodiments, contacting the one or more adipose progenitor cells with the isolated hydrogel composition is performed at a temperature ranging from about 6 degrees Celsius to about 40 degrees Celsius.

In some embodiments, contacting the one or more adipose progenitor cells with the isolated hydrogel composition is performed at a temperature of about 37 degrees Celsius.

In some embodiments, the isolated hydrogel composition is in a solid, gel, or semi-solid state.

In some embodiments, the step of implanting the isolated hydrogel composition comprising the one or more adipose progenitor cells into the subject comprises implanting hydrogel composition in a solid, gel, or semi-solid, isolated in the subject.

In some embodiments, the implanted isolated hydrogel composition does not cause one or more of an infectious disease, a bacterial infection, and an immune response in the subject after implantation.

In another aspect, the present disclosure provides methods of treating a subject. The methods can include obtaining a population of progenitor cells, optionally from the subject; optionally genetically altering one or more progenitor cells from the population of progenitor cells to correct an underlying genetic defect; optionally differentiating the one or more progenitor cells into differentiated cells; suspending the one or more progenitor cells in or contacting the one or more progenitor cells with any of the isolated hydrogel compositions disclosed herein, and implanting the isolated hydrogel composition comprising the one or more progenitor cells into the subject.

In some embodiments, suspending the one or more progenitor cells in the isolated hydrogel composition is performed at a temperature that is at least about 4 degrees Celsius or less.

In some embodiments, the isolated hydrogel composition is in a liquid or flowable state.

In some embodiments, wherein the step of implanting the one or more progenitor cells in the isolated hydrogel composition into the subject includes injecting the one or more progenitor cells and isolated hydrogel composition into the subject.

In some embodiments, contacting the one or more progenitor cells with the isolated hydrogel composition is performed at a temperature ranging from about 6 degrees Celsius to about 40 degrees Celsius.

In another aspect, the present disclosure provides methods of treating a subject. The methods can include obtaining a population of induced pluripotent stem cells (iPSCs); suspending the iPSCs in or contacting the iPSCs with any of the isolated hydrogel compositions disclosed herein; optionally genetically altering one or more iPSCs from the population; optionally differentiating the one or more iPSCs into differentiated cells; and implanting the isolated hydrogel composition comprising the differentiated cells into the subject.

In some embodiments, contacting the one or more progenitor cells with the isolated hydrogel composition is performed at a temperature of about 37 degrees Celsius.

In some embodiments, the isolated hydrogel composition is in a solid, gel, or semi-solid state.

In some embodiments, the step of implanting the isolated hydrogel composition comprising the one or more progenitor cells into the subject comprises implanting the hydrogel composition in a solid, gel, or semi-solid state in the subject.

In some embodiments, the implanted isolated hydrogel composition does not cause one or more of an infectious disease, a bacterial infection, and an immune response in the subject after implantation.

Unless otherwise defined, all technical and scientific terms used herein have the same meaning as commonly understood by one of ordinary skill in the art to which this invention belongs. Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The term “fragment,” as used herein, refers to a part or a segment of a larger molecule (e.g., the “fragment” can have a lower molecular weight than an intact molecule or a larger molecule).

The terms “subject” and “patient,” as used herein, refer to a mammal, including, but not limited to, a human or non-human mammal, such as a bovine, equine, canine, ovine, or feline mammal.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

As used herein, the terms “substantially free of,” “substantially all,” and the like refer to the hydrogel compositions being at least about 98% to about 100% (e.g., about 98% to about 98.5%, about 98.5% to about 99%, about 98.5% to about 100%, about 99% to about 99.5%, or about 99.5% to about 100%) free of the corresponding material and/or component described. For example, a hydrogel composition that is “substantially free of nucleic acids” refers to a hydrogel composition that is at least about 98% to about 100% free of nucleic acids. In another example, when “substantially all nucleic acids” have been removed from a hydrogel composition refers to at least about 98% to about 100% of the nucleic acids have been removed from the hydrogel composition.

The term “stem cell,” as used herein, refers to undifferentiated cells having a high proliferative potential with the ability to self-renew (e.g., to generate more stem cells via cell division) and to generate daughter cells that can undergo terminal differentiation into more than one distinct cell phenotype.

The term “pluripotent cell,” as used herein, refers to a cell having the ability to develop into multiple cells types, including all three embryonic lineages, forming the body organs, nervous system, skin, muscle, and skeleton.

The term “progenitor cell,” as used herein, refers to an early descendant of a stem cell that can only differentiate, but can no longer renew itself. Progenitor cells mature into precursor cells that mature into mature phenotypes.

The term “peptide,” as used herein, refers to two or more amino acids joined by a peptide bond.

The term “hydrogel,” as used herein, refers to a material that is not a readily flowable liquid nor a solid, but a gel in which water is the dispersion medium. Typically, a hydrogel comprises a plurality of polymer molecules that are cross-linked, either via covalent bonds or via non-covalent interactions, thereby forming a polymer scaffold, also referred to herein as a hydrogel scaffold. A hydrogel scaffold is typically super-absorbent, and a hydrogel can comprise more than about 99% water. Hydrogels useful in the context of this disclosure typically comprise a water content within the range of about 85% to about 99%. For example, in some embodiments, a hydrogel provided herein comprises a water content of about 99%, about 98%, about 97.5%, about 97%, about 96%, about 95%, about 94%, about 93%, about 92%, about 91%, or about 90%. In some embodiments, hydrogels with a water content of less than 90% are employed. A hydrogel may comprise components in addition to the scaffold and water, for example, cells, and/or drugs or compounds (e.g., growth factors) in a controlled-release form.

The term “hydrogel scaffold,” as used herein, refers to a water-insoluble network of polymers within a hydrogel.

The term “hAdipoGel,” as used herein refers to any of the isolated hydrogel compositions of the disclosure.

The term “semi-solid,” as used herein, refers to a viscous or highly viscous state.

As used herein, the term “optional” or “optionally” means that the subsequently described event or circumstance can or cannot occur, and that the description includes instances where the event or circumstance occurs and instances where it does not.

As used herein, the expression “pharmaceutically acceptable” applies to a composition that contains composition ingredients that are compatible with other ingredients of the composition as well as physiologically acceptable to the recipient (e.g., a mammal such as a human) without the resulting production of excessive undesirable and unacceptable physiological effects or a deleterious impact on the mammal being administered the pharmaceutical composition. A composition as described herein can comprise one or more carriers, useful excipients, and/or diluents.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Unless otherwise stated, the use of the term “about,” as used herein, refers to an amount that is above or below the stated amount by 10%. For example, “about” can mean a range including the particular value and ranging from 10% below that particular value and spanning to 10% above that particular value.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. It should be understood that various embodiments of the features of this disclosure are provided merely by way of example, and numerous variations, changes, and substitutions can occur to those skilled in the art without departing from the scope of this disclosure. It should also be understood that various alternatives to the specific embodiments described herein are also within the scope of this disclosure. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features and advantages of the invention will be apparent from the following detailed description and figures, and from the claims.

### DESCRIPTION OF DRAWINGS

FIG. 1 shows gel electrophoresis analysis of material extracted by guanidine hydrochloride (GuHCl) (labeled as “Extract”) and the final product (labeled as “hAdipoGel”). The final product (e.g., hAdipoGel) has many non-essential components removed.

FIG. 2 shows microscopy images of explanted human adipose tissue embedded in either Matrigel or hAdipoGel. Mesenchymal progenitor cells sprouted from the tissue after 5 days in culture in either Matrigel or hAdipoGel.

FIG. 3 shows microscopy images of mesenchymal progenitor cells produced from human explants in either Matrigel or hAdipoGel. The mesenchymal progenitor cells were plated and cultured with a differentiation medium to induce adipocyte differentiation. Accumulation of lipid was assessed by Oil Red-O staining.

FIG. 4 is a graph showing adipocytes from mesenchymal progenitor cells grown in hAdipoGel or Matrigel expressing similar markers. Mesenchymal progenitor cells grown in Matrigel (blue bars) or grown in two batches of hAdipoGel obtained from different donors (green and red bars) were plated and maintained in an undifferentiated state (C), induced to differentiate (M), or stimulated with forskolin after differentiation (F). The expression levels of genes are shown in the x-axis (Adiponectin, UCP1, or LINC00473).

FIGS. 5A-5B show mesenchymal progenitor cells growth in hAdipoGel maintain multipotency over more passages compared to Matrigel. FIG. 5A shows microscopy images of mesenchymal progenitor cells grown in Matrigel (top row) or in two batches of hAdipoGel obtained from different donors (middle and bottom rows). The mesenchymal progenitor cells were plated, allowed to reach confluence, and passaged at a 1:2 ratio for the number of passages indicated in FIG. 5A. At each passage, the capacity of cells to differentiate into adipocytes was assessed by Bodipy (green) staining. FIG. 5B shows a graph illustrating the quantification of Bodipy staining at each passage.

FIGS. 6A-6B show mesenchymal progenitor cells resuspended in hAdipoGel or Matrigel and implanted into nude mice forming equally functional tissue. Mesenchymal progenitor cells were resuspended in either Matrigel or hAdipoGel and implanted subcutaneously into immunocompromised NSG mice. After 10 weeks, tissue formed from implanted cells was excised and analyzed by histochemistry. FIG. 6A shows photographs of the excised tissue (top row) and microscopy images of the histochemical staining of the excised tissue (bottom row). Images and histochemical analysis results were comparable between conditions. Serum from mice implanted with cell-loaded Matrigel (“Matrigel”), cell-loaded hAdipoGel (“hAdipoGel”), and control gel (“Matrigel No Cells”) was analyzed for human adiponectin content. FIG. 6B is a graph showing the adiponectin content in these three conditions (i.e., “Matrigel,” “hAdipoGel,” and “Matrigel No Cells”). Production of adiponectin from cells implanted in Matrigel or in hAdipoGel was statistically indistinguishable.

FIGS. 7A-7D show production levels of C-peptide produced by induced pluripotent stem cell (iPSC)-derived beta cells in mice. FIG. 7A is a graph showing the levels of C-peptide produced by iPSC-derived beta cells encapsulated within an hAdipoGel substrate implanted subcutaneously in mice. FIG. 7B is a graph showing the levels of C-peptide produced by iPSC-derived beta cells implanted within a subrenal cavity (e.g., a kidney capsule) in mice. FIG. 7C is a graph showing ratios of the levels of C-peptide produced by iPSC-derived beta cells after/before a glucose injection in mice; the iPSC-derived beta cells were encapsulated within an hAdipoGel substrate implanted subcutaneously in mice. FIG. 7D is a graph showing ratios of the levels of C-peptide produced by iPSC-derived beta cells after/before a glucose injection in mice; the iPSC-derived beta cells were implanted within a subrenal cavity (e.g., a kidney capsule) in mice.

### DETAILED DESCRIPTION

A major hurdle for cell-based therapies is the paucity of three-dimensional scaffolds, which are needed to culture cells in vitro prior to implantation in the body, that are compatible with Good Manufacturing Practices (GMP). Scaffolds provide crucial extracellular signals that cells require for proper proliferation and maintenance of function. Currently, the best scaffolds for cell therapy research are typically derived from animal cells (e.g., mouse sarcoma cells). Therefore, current scaffolds are incompatible with clinical use due to the risk of transmitting pathogens and the introduction of tumor-derived growth factors. While other chemical hydrogels have been developed, none are as effective as scaffolds derived from animal cells, nor can they be scaled for broad clinical use.

Existing and emerging solutions for producing scaffolds for stem/progenitor cell therapies all attempt to mimic the superior properties of biological matrices, the benchmark being hydrogels extracted from mouse sarcomas, which are incompatible with clinical use. Other attempts include the use of feeder cell layers, or synthetic or scaffolds (e.g., an acrylate surface with deposits of various peptide-polymer conjugates). Laminins provide some of the biologically relevant signals but are expensive to produce. Synthetic or chemically-derived scaffolds fail to provide other signals from the extracellular matrix that are critical for preventing genetic and phenotypic drift in vitro. Therefore, there is a need for three-dimensional scaffolds or hydrogels that mimic the properties of biological matrices while fulfilling GMP requirements and being amenable for scale-up manufacturing processes.

This disclosure provides biological three-dimensional scaffolds or hydrogels (also referred herein as “hAdipoGel”), which are extracted and processed from human adipose tissue (e.g., surgically discarded adipose tissue). Embodiments may provide one or more of the following advantages. In some embodiments, the hydrogel compositions described herein provide the critical properties of the extracellular matrix, while being compatible with Good Manufacturing Practices and inexpensive to produce.

In some embodiments, the hydrogel compositions of the disclosure are superior to synthetic scaffolds and/or animal-derived scaffolds in supporting human mesenchymal progenitor cell proliferation with maintenance of multipotency. For example, in some embodiments, the hydrogel compositions of the disclosure are superior than animal-derived and synthetic scaffolds and/or hydrogels in supporting human stem/progenitor cell growth, human stem/progenitor cell engineering, and human stem/progenitor cell implantation in humanized mice.

In some embodiments, the methods disclosed herein are advantageously reproducible and generate a high-yield of the hydrogel compositions. For example, in some embodiments, the hydrogel compositions have high reproducibility (as assessed by mass spectrometry and biological function) between samples derived from different patients. In some embodiments, the hydrogel compositions of the disclosure are superior to animal-derived and synthetic scaffolds and/or hydrogels in supporting proliferation of human mesenchymal progenitor/stem cells, and supporting multilineage differentiation (e.g., adipogenic, osteogenic, and chondrogenic differentiation).

In some embodiments, yet another advantage of the methods of the disclosure is that the methods described herein may be scalable and may involve low manufacturing costs, as compared to synthetic and/or animal-derived scaffolds and/or hydrogels. For example, the human adipose tissue used in the methods of the disclosure can be sourced from surgically discarded human adipose tissue. In some embodiments, the reagents and equipment used in the methods disclosed herein are inexpensive and/or commonly found in laboratory and/or manufacturing environments.

### Isolated Hydrogel Compositions

As demonstrated herein (e.g., in Example 1), the present disclosure features isolated hydrogel compositions derived from human adipose tissues. The isolated hydrogel compositions can include one or more (i.e., 1, 2, 3, 4, 5, or all) of collagen 1A1 (COL1A1), collagen 1A2 (COL1A2), collagen 3A1 (COL3A1), collagen 4A2 (COL4A2), collagen 5A2 (COL5A2), and fibrillin-1 (FBN1).

One or more components of the isolated hydrogel compositions (e.g., comprising COL1A1, COL1A2, COL3A1, COL4A2, COL5A2, and FBN1) can be fragmented after the methods of producing the hydrogel are completed. In some embodiments, the isolated hydrogel compositions include fragments of proteins (e.g., polypeptides), fragments of polypeptides (e.g., peptides), and/or fragments of peptides (e.g., amino acids). For example, the hydrogel compositions can include fragments of one or more of COL1A1, COL1A2, COL3A1, COL4A2, COL5A2, and FBN1 (e.g., polypeptides), fragments of polypeptides (e.g., peptides), and/or fragments of peptides (e.g., amino acids). In some embodiments, one or more steps of the methods of preparing the isolated hydrogel compositions can cause the fragmentation of proteins (e.g., polypeptides), polypeptides (e.g., peptides), and/or peptides (e.g., amino acids) in the starting human adipose tissue from which the isolated hydrogel composition is derived. For example, in some embodiments, the fragmentation of proteins (e.g., polypeptides), polypeptides (e.g., peptides), and/or peptides (e.g., amino acids) in the starting human adipose tissue can be caused by the slicing of the adipose tissue sample into a sheet, the contacting of the adipose tissue sample with a denaturant, the mechanically processing of the adipose tissue sample, the contacting of the adipose tissue sample with a nuclease, the contacting of the adipose tissue sample with an organic solvent, the contacting of the adipose tissue sample with a protease, or any combinations thereof. In some embodiments, the size of the fragments of one or more of COL1A1, COL1A2, COL3A1, COL4A2, COL5A2, and FBN1 ranges from about at least 10 kilodalton (kDa) to about 30 kDa (e.g., about 10 kDa to about 15 kDa, about 10 kDa to about 20 kDa, about 10 kDa to about 25 kDa, about 10 kDa to about 30 kDa, about 15 kDa to about 20 kDa, about 15 kDa to about 25 kDa, about 15 kDa to about 30 kDa about 20 kDa to about 25 kDa, about 20 kDa to about 30 kDa, or about 25 kDa to about 30 kDa). In some embodiments, the size of the fragments of one or more of COL1A1, COL1A2, COL3A1, COL4A2, COL5A2, and FBN1 is about 20 kDa.

In some embodiments, the fragment sizes are polydisperse and vary in size. In some embodiments, the fragment sizes are substantially monodisperse and have a substantially uniform size. In some embodiments, the isolated hydrogel compositions are substantially free of intact COL1A1, COL1A2, COL3A1, COL4A2, COL5A2, and/or FBN1 (e.g., collagen and/or fibrillin-1 having an intact, native, or non-denatured protein structure). In some embodiments, the isolated hydrogel compositions are at least about 98% to about 100% (e.g., about 98% to about 98.5%, about 98.5% to about 99%, about 98% to about 100%, about 99% to about 99.5%, or about 99.5% to about 100%) free of intact COL1A1, COL1A2, COL3A1, COL4A2, COL5A2, and/or FBN1 (e.g., collagen and/or fibrillin-1 having an intact, native, or non-denatured protein structure). In some embodiments, the fragments are biologically active fragments. In some embodiments, the isolated hydrogel composition is a defined mixture of fragments of specific sizes from COL1A1, COL1A2, COL3A1, COL4A2, COL5A2, and FBN1. In some embodiments, the isolated hydrogel compositions do not include all of the native extracellular matrix (ECM) proteins found in a mammalian adipose tissue.

In some embodiments, the COL1A1 is present in the isolated hydrogel composition at a concentration that is higher than each of the concentrations of COL1A2, COL3A1, COL4A2, COL5A2, and/or FBN1. In some embodiments, the COL1A2 is present in the isolated hydrogel composition at a concentration that is higher than each of the concentrations of COL3A1, COL4A2, COL5A2, and/or FBN1. In some embodiments, the COL3A1 is present in the isolated hydrogel composition at a concentration that is higher than each of the concentrations of COL4A2, COL5A2, and/or FBN1. In some embodiments, the COL4A2 is present in the isolated hydrogel composition at a concentration that is higher than each of the concentrations of COL5A2 and/or FBN1. In some embodiments, the COL5A2 is present in the isolated hydrogel composition at a concentration that is higher than FBN1. In some embodiments, FBN1 is present in a concentration that is less than each of a concentration of collagen 1A1, collagen 1A2, collagen 3A1, collagen 4A2, and collagen 5A2.

In some embodiments, collagen 1A1 is present at a concentration of about 20 weight (wt) %, collagen 1A2 is present at a concentration of about 18 wt %, collagen 3A1 is present at a concentration of about 17 wt %, collagen 4A2 is present at a concentration of about 16 wt %, collagen 5A2 is present at a concentration of about 15 wt %, and fibrillin-1 is present at a concentration of about 14 wt %.

In some embodiments, collagen 1A1 is present in the hydrogel composition at a concentration ranging from about 15 wt % to about 25 wt % (e.g., about 15 wt % to about 20 wt % or about 20 wt % to about 25 wt %). In some embodiments, collagen 1A1 is present in the hydrogel composition at a concentration ranging from about at least 1 wt % to about 99 wt % (e.g., about 1 wt % to about 5 wt %, about 5 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, about 90 wt % to about 95 wt %, about 95 wt % to about 99 wt %).

In some embodiments, collagen 1A2 is present in the hydrogel composition at a concentration ranging from about 13 wt % to about 23 wt %. In some embodiments, collagen 1A2 is present in the hydrogel composition at a concentration ranging from about at least 1 wt % to about 99 wt % (e.g., about 1 wt % to about 5 wt %, about 5 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, about 90 wt % to about 95 wt %, about 95 wt % to about 99 wt %).

In some embodiments, collagen 3A1 is present in the hydrogel composition at a concentration ranging from about 12 wt % to about 22 wt %. In some embodiments, collagen 3A1 is present in the hydrogel composition at a concentration ranging from about at least 1 wt % to about 99 wt % (e.g., about 1 wt % to about 5 wt %, about 5 wt % to about 10 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, about 90 wt % to about 95 wt %, about 95 wt % to about 99 wt %).

In some embodiments, collagen 4A2 is present in the hydrogel composition at a concentration ranging from about 11 wt % to about 21 wt %. In some embodiments, collagen 4A2 is present in the hydrogel composition at a concentration ranging from about at least 1 wt % to about 99 wt % (e.g., about 1 wt % to about 5 wt %, about 1 wt % to about 10 wt %, about 1 wt % to about 15 wt %, about 1 wt % to about 20 wt %, about 1 wt % to about 25 wt %, about 5 wt % to about 10 wt %, about 5 wt % to about 15 wt %, about 5 wt % to about 20 wt %, about 5 wt % to about 25 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, about 90 wt % to about 95 wt %, about 95 wt % to about 99 wt %).

In some embodiments, collagen 5A2 is present in the hydrogel composition at a concentration ranging from about 10 wt % to about 20 wt %. In some embodiments, collagen 5A2 is present in the hydrogel composition at a concentration ranging from about at least 1 wt % to about 99 wt % (e.g., about 1 wt % to about 5 wt %, about 1 wt % to about 10 wt %, about 1 wt % to about 15 wt %, about 1 wt % to about 20 wt %, about 1 wt % to about 25 wt %, about 5 wt % to about 10 wt %, about 5 wt % to about 15 wt %, about 5 wt % to about 20 wt %, about 5 wt % to about 25 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, about 90 wt % to about 95 wt %, about 95 wt % to about 99 wt %).

In some embodiments, fibrillin-1 is present in the hydrogel composition at a concentration ranging from about 9 wt % to about 19 wt %. In some embodiments, fibrillin-1 is present in the hydrogel composition at a concentration ranging from about at least 1 wt % to about 99 wt % (e.g., about 1 wt % to about 5 wt %, about 1 wt % to about 10 wt %, about 1 wt % to about 15 wt %, about 1 wt % to about 20 wt %, about 1 wt % to about 25 wt %, about 5 wt % to about 10 wt %, about 5 wt % to about 15 wt %, about 5 wt % to about 20 wt %, about 5 wt % to about 25 wt %, about 10 wt % to about 15 wt %, about 15 wt % to about 20 wt %, about 20 wt % to about 25 wt %, about 25 wt % to about 30 wt %, about 30 wt % to about 35 wt %, about 35 wt % to about 40 wt %, about 40 wt % to about 45 wt %, about 45 wt % to about 50 wt %, about 50 wt % to about 55 wt %, about 55 wt % to about 60 wt %, about 60 wt % to about 65 wt %, about 65 wt % to about 70 wt %, about 70 wt % to about 75 wt %, about 75 wt % to about 80 wt %, about 80 wt % to about 85 wt %, about 85 wt % to about 90 wt %, about 90 wt % to about 95 wt %, about 95 wt % to about 99 wt %).

In some embodiments, the isolated hydrogel composition is substantially free of native cells or endogenous cells. In some embodiments, the isolated hydrogel composition is substantially free of cells. In some embodiments, the isolated hydrogel composition is substantially free of nucleic acids (e.g., DNA and/or RNA) or endogenous nucleic acids. In some embodiments, the isolated hydrogel composition is substantially free of non-fibrous proteins or endogenous non-fibrous proteins. In some embodiments, the isolated hydrogel composition is substantially free of globular proteins or endogenous globular proteins. In some embodiments, the isolated hydrogel composition is substantially free of lipids or endogenous lipids. In some embodiments, the isolated hydrogel composition is substantially free of entactins (e.g., nidogens) or endogenous entactins. In some embodiments, the isolated hydrogel composition is substantially free of laminin or endogenous laminin. In some embodiments, the isolated hydrogel composition is substantially free of proteoglycans (e.g., heparan sulfate proteoglycan) or endogenous proteoglycans. In some embodiments, the isolated hydrogel composition is substantially free of pathogens. In some embodiments, the isolated hydrogel composition is substantially free of substances that would pose a risk for transmission of an infectious disease and/or microbial contamination when implanted in a patient. In some embodiments, the isolated hydrogel composition is substantially free of process-related impurities that may pose a risk for a patient being treated with the isolated hydrogel composition.

In some embodiments, the isolated hydrogel composition forms a gel, solidifies, and/or polymerizes when exposed to a temperature ranging from about 37 degrees Celsius to about 40 degrees Celsius. For example, in some embodiments, one or more of collagen 1A1, collagen 1A2, collagen 3A1, collagen 4A2, collagen 5A2, and fibrillin-1 found in the isolated hydrogel composition polymerizes when exposed to a temperature ranging from about 37 degrees Celsius to about 40 degrees Celsius. In some embodiments, the isolated hydrogel composition forms a gel, solidifies, and/or polymerizes when exposed to a temperature of about 37 degrees Celsius. In some embodiments, the isolated hydrogel composition is not in an injectable form when exposed to a temperature of about 37 degrees Celsius.

In some embodiments, the isolated hydrogel composition is a liquid state when exposed to a temperature ranging from about 1 degree Celsius to about 5 degrees Celsius.

In some embodiments, the isolated hydrogel composition is a liquid state when exposed to a temperature of about 4 degrees Celsius. In some embodiments, the isolated hydrogel composition is in an injectable form when exposed to a temperature of about 4 degrees Celsius.

In some embodiments, the once the hydrogel composition has been isolated, one or more exogenous components may be added. In some embodiments, the exogenous component is a purified or isolated component. In some embodiments, the exogenous component is a therapeutic agent (e.g., a drug delivery payload). In some embodiments, the therapeutic agents are encapsulated in, carried by, or otherwise loaded in or on the hydrogel compositions. In some embodiments, one or more therapeutic agents are dispersed, embedded, suspended, and/or mixed within the composition.

In some embodiments, the exogenous component is a growth factor. In some embodiments, the growth factor is a pro-angiogenic factor. Pro-angiogenic factors include growth factors and components that support the growth of cells that compose blood vessels. In some embodiments, the pro-angiogenic factors include basic fibroblast growth factor (FGF-2), vascular endothelial growth factor (VEGF), insulin-like growth factor-1 (IGF1), and/or epidermal growth factor (EGF). In some embodiments, the growth factor is a human growth factor. In some embodiments, the growth factor is a growth factor analog.

In some embodiments, the exogenous component is an antibiotic. In some embodiments, the exogenous component is an anti-inflammatory agent. In some embodiments, the isolated hydrogel composition further includes a carrier or excipient. In some embodiments, the carrier or excipient is exogenous. The carrier or excipient can be a pharmaceutically acceptable inert agent or vehicle for delivering one or more active agents present in the hydrogel compositions to a patient. In some embodiments, the isolated hydrogel composition includes water. In some embodiments, the exogenous component is a cell (e.g., a mammalian cell). In some embodiments, the exogenous component is an induced pluripotent stem cell (iPSC). In some embodiments, the exogenous component is an iPSC-derived beta cell.

In some embodiments, the composition further includes a pharmaceutically acceptable carrier. As used herein, the expression “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS).

### Methods of Preparing Isolated Hydrogel Compositions

Provided herein are methods of preparing an isolated hydrogel composition. The method includes providing or using an adipose tissue sample from a subject. In some embodiments, the adipose tissue is a human adipose tissue. In some embodiments, the subject is a live human subject. In some embodiments, the subject is a cadaver. In some embodiments, the subject is a non-human subject. The adipose tissue can be obtained using methods known in the art, e.g., by needle biopsy, surgical harvesting (e.g., after a panniculectomy), or lipoaspiration. In some embodiments, the adipose tissue is a surgically discarded adipose tissue.

Next, the methods can include freezing the adipose tissue sample. In some embodiments, the adipose tissue sample is frozen, e.g., at about −20 degrees Celsius. In some embodiments, the adipose tissue sample is exposed to a temperature ranging from at least about −25 degrees Celsius to about 0 degrees Celsius (e.g., from about −25 degrees Celsius to about −20 degrees Celsius, from about −20 degrees Celsius to about −15 degrees Celsius, from about −20 degrees Celsius to about −10 degrees Celsius, from about −20 degrees Celsius to about −5 degrees Celsius, or from about −20 degrees Celsius to about 0 degrees Celsius) for a sufficient period of time (e.g., until the adipose tissue freezes).

Once the adipose tissue sample is frozen, the methods include slicing the sample into sheets (e.g., thin, flat sections) while it is still frozen. In some embodiments, the sheets have substantially uniform thickness. In some embodiments, the sheets have a thickness ranging from at least about 1 millimeter (mm) to about 4 mm (e.g., from about 1 mm to about 3 mm, about 1.5 mm to about 3 mm, about 2 mm to about 3 mm, about 2.5 mm to about 3 mm, about 3 mm to about 3.5 mm, or about 3 mm to about 4 mm). In some embodiments, the sheets have a thickness of about 3 mm.

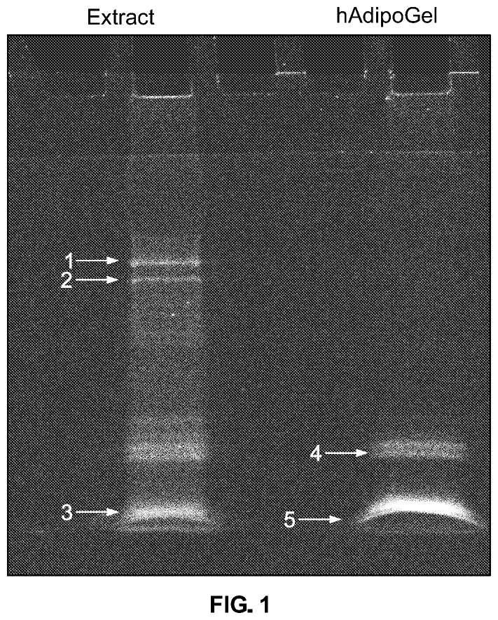

Next, the methods include contacting the sample (e.g., the thin sheets) with a denaturant to denature substantially all non-fibrous protein in the sample. In some embodiments, the sample (e.g., the thin sheets) is contacted with the denaturant while it is still frozen. In some embodiments, the sample (e.g., the thin sheets) is contacted with the denaturant once it has been thawed. In some embodiments, the denaturant is guanidine hydrochloride, as shown in FIG. 1. In some embodiments, the denaturant is urea. In some embodiments, the denaturant is acetonitrile. In some embodiments, the denaturant is an aqueous solution of an inorganic or an organic salt (e.g., urea, sodium dodecyl sulfate, sodium sulphite, guanidine hydrochloride, or the like). In some embodiments, the denaturant is a fluorinated solvent, an ionic liquid, a strong acid, or any combination thereof. In some embodiments, the concentration of the denaturant is about 4 molar (M). In some embodiments, the concentration of the denaturant ranges from at least about 1 M to about 5 M (e.g., about 1 M to about 4 M, about 1.5 M to about 4 M, about 2 M to about 4 M, about 2.5 M to about 4 M, about 3 M to about 4 M, about 3.5 M to about 4 M, about 4 M to about 4.5 M, or about 4 M to about 5 M). In some embodiments, the methods include contacting the sample with a denaturant at a temperature of about 4 degrees Celsius. In some embodiments, the methods include contacting the sample with a denaturant at a temperature ranging from at least about 1 degree Celsius to about 7 degrees Celsius (e.g., about 1 degree Celsius to about 4 degrees Celsius, about 2 degree Celsius to about 4 degrees Celsius, about 3 degrees Celsius to about 4 degrees Celsius, about 4 degrees Celsius to about 5 degrees Celsius, about 4 degrees Celsius to about 6 degrees Celsius, or about 4 degrees Celsius to about 7 degrees Celsius). In some embodiments, the methods include incubating the sample with a denaturant for at least 72 hours. In some embodiments, the methods include incubating the sample with a denaturant for at least about 24 hours to about 120 hours (e.g., about 24 hours to about 72 hours, about 48 hours to about 72 hours, 72 hours to about 96 hours, or 72 hours to about 120 hours). After incubation with the denaturant, the methods include removing the denaturant and washing the adipose tissue sample in water. The washing step can include gentle mixing. In some embodiments, the washing step is performed at about 4 degrees Celsius for about 1 hour.

Then, the methods include mechanically processing the sample to lyse substantially all cells and cellular material in the sample. In some embodiments, mechanically processing the sample includes homogenizing the sample. In some embodiments, mechanically processing the sample includes mechanically disrupting and/or triturating the sample. In some embodiments, the methods include adding water to the sample prior to mechanically processing the sample. In some embodiments, about 30 milliliters (ml) of water is added to about 2.5 grams (g) of adipose tissue sample. In some embodiments, the water is cold (e.g., ranging from about 1 degree Celsius to about 4 degrees Celsius). In some embodiments, the sample is mechanically processed using a rotor-stator homogenizer. After the adipose tissue sample is mechanically processed, the sample is subjected to centrifugation and the pelleted material is saved.

Next, the methods include contacting the sample (e.g., the pelleted material) with a nuclease to remove substantially all nucleic acid material from the sample. In some embodiments, the nuclease is an endonuclease. In some embodiments, the nuclease includes one or more of DNase1, RNaseA, magnesium chloride, and phosphate buffered saline (PBS). In some embodiments, the sample is incubated with the nuclease for at least about 1 hour. In some embodiments, the sample is incubated with the nuclease for at least about 0.5 hours to about 2 hours (e.g., 0.5 hours to about 1 hour, 1 hour to about 1.5 hours, or about 1 hours to about 2 hours). In some embodiments, the sample is contacted with the nuclease at a temperature of about 37 degrees Celsius. Next, in some embodiments, after the sample is incubated with the nuclease, the sample is washed (e.g., using a cell strainer) with water. In some embodiments, the method does not include contacting the sample with a detergent. In some embodiments, substantially all nucleic acids are removed from the composition prior to removal of substantially all lipids from the composition.

Then, the methods include contacting the sample with an organic solvent to remove substantially all lipids from the sample. In some embodiments, the organic solvent is polar. In some embodiments, the organic solvent is non-polar. In some embodiments, the organic solvent is ethanol, methanol, chloroform, or any combination thereof. In some embodiments, the method includes incubating the sample with ethanol for about 15 minutes at a temperature of about 37 degrees Celsius. In some embodiments, the method includes incubating the sample with a mixture of chloroform and methanol for about 20 minutes at room temperature (e.g., about 23 degrees Celsius). In some embodiments, the mixture of chloroform and methanol includes a chloroform-to-methanol ratio of about 2:1. In some embodiments, the mixture of chloroform and methanol includes a chloroform-to-methanol ratio of about 4:1 to about 1:4 (e.g., about 4:1 to about 2:1, about 3:1 to about 2:1, about 2:1 to about 1:1, about 2:1 to about 1:2, about 2:1 to about 1:3, about 2:1 to about 1:4). In some embodiments, after the sample is incubated with the organic solvents (e.g., with ethanol and/or the mixture of chloroform and methanol, the sample is washed (e.g., using a cell strainer) with water.

Next, the methods include contacting the sample with a protease to digest proteins in the sample. In some embodiments, the protease is pepsin. In some embodiments, the protease includes one or more of trypsin, chymotrypsin, dispase, collagenase, and thermolysin. In some embodiments, the protease is combined with an acid in solution (e.g., protease-acid solution). In some embodiments, the acid is a weak acid. In some embodiments, the acid has a pH ranging from about 5 to about 7. In some embodiments, the acid is acetic acid. In some embodiments, the acid is one or more of peracetic acid (PAA), hydrochloric acid, and sulfuric acid. In some embodiments, the protease comprises a protease-acid solution having a weight that is about 4 times the weight of the sample (e.g., the weight of the sample at this stage in the process). In some embodiments, the protease comprises a protease-acid solution having a weight that is about at least 1 time to about 7 times (e.g., about 1 time to about 4 times, about 2 times to about 4 times, about 3 times to about 4 times, about 4 times to about 5 times, about 4 times to about 6 times, or about 4 times to about 7 times) the weight of the sample. In some embodiments, the methods include weighing the sample prior to contacting the sample with the protease. In some embodiments, the concentration of the protease in the protease-acid solution is about 75% by weight. In some embodiments, the concentration of the protease in the protease-acid solution is about 75% by volume. In some embodiments, the acid is the concentration of the acid in the protease-acid solution is about 0.5 M. In some embodiments, the methods include submerging the sample in acetic acid prior to contacting the sample with the protease. In some embodiments, the methods include contacting the sample with the protease at room temperature (e.g., about 23 degrees Celsius). In some embodiments, the methods include contacting the sample with the protease for at least about 48 hours. In some embodiments, the methods include contacting the sample with the protease for at least about 12 hours to about 72 hours (e.g., about 12 hours to about 48 hours, about 36 hours to about 48 hours, about 48 hours to about 60 hours, about 48 hours to about 72 hours).

Then, the methods include centrifuging the material and collecting the supernatant. After centrifuging, the methods include dialyzing the sample using a dialysis membrane. In some embodiments, the dialysis membrane has a molecular weight cutoff ranging from about 12 kDa to about 14 kDa. In some embodiments, the dialysis membrane has a molecular weight cutoff ranging from about 10 kDa to about 16 kDa (e.g., about 10 kDa to about 12 kDa, about 11 kDa to about 12 kDa, about 12 kDa to about 13 kDa, about 12 kDa to about 14 kDa, about 12 kDa to about 15 kDa, about 12 kDa to about 16 kDa). In some embodiments, the sample is sterilized by dialysis. In some embodiments, the sample is dialyzed against a buffer (e.g., Tris-buffered saline). In some embodiments, the sample is dialyzed against a buffer having a volume of about 100 times the volume of the sample. In some embodiments, the sample is dialyzed for about 24 hours. In some embodiments, the sample is dialyzed for about 16 hours. In some embodiments, the sample is dialyzed one or more times against one or more different dialysis reagents. In some embodiments, the dialysis reagent is urea, chloroform, phosphate buffered saline, and/or Tris-buffered saline (TBS). In some embodiments, after the sample is dialyzed against a buffer for a first time, the sample is then dialyzed one, two, three, four or more subsequent times. In some embodiments, after the sample is dialyzed against a buffer for a first time, the sample is then dialyzed one, two, three, four or more subsequent times for about 16 hours. In some embodiments, after the sample is dialyzed against a buffer for a first time, the sample is then dialyzed one, two, three, four or more subsequent times at a temperature of about 4 degrees Celsius. In some embodiments, after the sample is dialyzed against a buffer for a first time, the sample is then dialyzed against urea. Next, in some embodiments, the sample is dialyzed against chloroform mixed in with TBS. In some embodiments, the concentration of chloroform in the mixture is about 0.5%. Next, in some embodiments, the sample is dialyzed against TBS. Next, in some embodiments, the sample is dialyzed against phosphate buffered saline (PBS). Next, in some embodiments, the sample is collected and stored at about −20 degrees Celsius.

In some embodiments, the methods disclosed herein can be used to generate a high yield of isolated hydrogel compositions. For example, in some embodiments, the yield is about 50%. For example, in some embodiments, the yield ranges from at least about 40% to about 95% (e.g., about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, or about 50% to about 95%). In some embodiments, the yield is about 75%. In some embodiments, the yield is about 85%. In some embodiments, the yield is about 90%.

In some embodiments, the steps of the methods of preparation of isolated hydrogel compositions do not need to be performed in the order that they were described in.

### Methods of Use

The isolated hydrogel compositions described herein can be used, e.g., to culture cells in a three-dimensional environment. For example, the isolated hydrogel compositions can be used to culture stem cells or undifferentiated cells. In some embodiments, the isolated hydrogel compositions can be used to culture induced pluripotent stem cells (iPSCs). In some embodiments, the isolated hydrogel compositions can be used to culture iPSC-derived beta cells. iPSCs are typically derived by introducing a specific set of pluripotency-associated genes, or “reprogramming factors,” into an adult cell type. These cells show qualities very similar to human embryonic stem cells. The original set of reprogramming factors (also called Yamanaka factors) are the genes Oct4 (Pou5f1), Sox2, cMyc, and Klf4. Multiple methods can be used to generate iPSCs, including, but not limited to, retrovirus or lentivirus-mediated gene transduction and chemical induction. The retroviral vectors require integration into host chromosomes to express reprogramming genes, but DNA-based vectors and plasmid vectors do not generally integrate to the cell genome. In some embodiments, to generate the iPSCs, each of the pluripotency factors can be also replaced by related transcription factors, miRNAs or small molecules. After introduction of reprogramming factors, cells begin to form colonies very similar to human embryonic stem cells. In some embodiments, these iPSC colonies can be isolated based on their morphology, expression of pluripotent genes and surface markers, and can be expanded in an appropriate culture system (e.g., the hAdipoGel compositions of the disclosure) to keep pluripotency over several passages. In some embodiments, the methods of the disclosure are feeder-free methods that do not require the use of mouse or human fibroblast feeder layers and, instead, use the hAdipoGel compositions described herein.

In some embodiments, the isolated hydrogel compositions can be used to simultaneously culture one or more types of cells. In some embodiments, the stem cells cultured in the isolated hydrogel compositions of the disclosure retain multipotency for a longer period of time than an animal-derived hydrogel. In some embodiments, the isolated hydrogel compositions can be used as a scaffold in tissue engineering or cell therapy applications, wherein cells (e.g., iPSC-derived beta cells) to be implanted are suspended in the hydrogel compositions before being administered to a subject in need thereof.

In some embodiments, the isolated hydrogel compositions provide the necessary environment for long-term in vivo and/or in vitro culture (e.g., about 4 weeks to about 12 weeks or more) of cells. For example, as shown in Example 2, the isolated hydrogel compositions can be used to culture iPSC-derived beta cells in vivo. In some embodiments, the embedded and/or seeded cells within the isolated hydrogel compositions maintain functionality. In some embodiments, the isolated hydrogel compositions can include one or more of the exogenous components described elsewhere herein (e.g., growth factors, antibiotic agents, therapeutic agents, anti-inflammatory agents, or the like) in addition to one or more cell types.

As another example, the isolated hydrogel compositions described herein can be used to treat subjects. For example, the isolated hydrogel compositions can be used to treat subjects who need reconstruction of soft tissues or who have cartilage and mucogingival tissue defects. In some embodiments, the isolated hydrogel compositions can be used for cosmetic applications (e.g., as a dermal filler). In some embodiments, the isolated hydrogel compositions can be used in the manufacturing or production of cell-based therapies for the treatment and/or prevention of various diseases, as described elsewhere herein. For example, the isolated hydrogel compositions can be administered to subjects having lipodystrophy by supporting the ex vivo production of progenitor cells from these patients, correcting the underlying genetic defect, differentiating into adipocytes, and implanting the differentiated cells back into the patient where they can continue to form healthy adipose tissue.

In some embodiments, the methods of treatment include obtaining a population of progenitor cells. In some embodiments, the population of progenitor cells is a population of adipose progenitor cells and/or mesenchymal progenitor cells. In some embodiments, the population of progenitor cells is obtained from the patient. In some embodiments, the population of progenitor cells is obtained from a donor. Next, in some embodiments, one or more progenitor cells from the population of progenitor cells can be optionally genetically altered. In some embodiments, one or more progenitor cells can be genetically altered to correct an underlying genetic defect. In some embodiments, the genetic defect is a genetic mutation. In some embodiments, the genetic defect is a lipodystrophy genetic mutation. Next, in some embodiments, the methods include optionally differentiating the progenitor cells into differentiated cells. In some embodiments, the differentiated cells are adipocytes. In some embodiments, the methods include optionally differentiating mesenchymal progenitor cells into adipocytes.

Next, in some embodiments, the methods include suspending the progenitor cells or the differentiated cells in or contacting the progenitor cells or the differentiated cells with any of the isolated hydrogel composition described herein. For example, in some embodiments, the progenitor cells and/or the differentiated cells can be mixed in and suspended in the isolated hydrogel compositions described herein when the isolated hydrogel composition is in a liquid state (e.g., at a temperature of about 1° C. to about 5° C.). In some embodiments, the progenitor cells and/or the differentiated cells can be seeded on top of the isolated hydrogel composition when the isolated hydrogel composition is in a gel, solid, or semi-solid state (e.g., at a temperature of about 37° C. to about 40° C.).

Next, in some embodiments, the methods include implanting the isolated hydrogel composition comprising the progenitor cells or the differentiated cells in the subject. For example, in some embodiments, the isolated hydrogel composition comprising the progenitor cells or the differentiated cells can be injected into the subject when the composition is in an injectable and/or liquid state. In another example, in some embodiments, the isolated hydrogel composition comprising the progenitor cells or the differentiated cells can be implanted into the subject when the composition is in a gel, solid, or semi-solid state.

In some embodiments, the methods include obtaining a population of iPSCs, suspending the iPSCs in or contacting the iPSCs with any of the isolated hydrogel compositions disclosed herein, optionally genetically altering one or more iPSCs from the population of iPSCs, differentiating the one or more iPSCs into differentiated cells, and implanting the isolated hydrogel composition comprising the differentiated cells into the subject. In some embodiments, the differentiated cells are iPSC-derived beta cells.

### EXAMPLES

The invention is further described in the following examples, which do not limit the scope of the invention described in the claims.

### Example 1—Preparation of hAdipoGels

hAdipoGel is a hydrogel formulation obtained from surgically discarded human adipose tissue. Briefly, the process to prepare hAdipoGel involved freezing discarded human adipose tissue obtained from the operating room to −20° C. Next, about 10 g of tissue was then sliced into 2-3 mm thick slices. Next, the slices of tissue were incubated in 4M Guanidine HCl extraction buffer for 72 h at 4 degrees (minimum of 2× volume). Guanidine HCL (Soluble protein) was then removed and tissue slices were washed in water for 1 h at 4 degrees while gently mixing. The water was changed every 10 minutes. Next, the tissue sample was homogenized (2.5 g/tissue in 30 ml cold water). Next, the tissue sample was centrifuged at 1200×g for 5 min. The pelleted material was saved. The top layers were collected into a new tube containing 30 ml of chilled water and the homogenization/spin steps were repeated. Next, the pelleted material from both rounds of homogenization/spin steps was combined, weighed, and incubated in 1.5×(w/v) nuclease buffer for 1 h at 37 degrees Celsius. 10 ml of nuclease buffer included 100 μl of 5 mg/ml DNase1, 10 μl of 10 mg/ml RNaseA, 100 μl 1M MgCl₂, and 9.79 ml phosphate buffered saline (PBS). Next, the material was washed using a filter unit (e.g., a cell strainer) with 30× volume water. Next, the material was removed material from the filter and incubated in >10× volume of 70% ethanol for 15 min at 37 degrees Celsius while mixing simultaneously. Next, the material was washed using a filter unit (e.g., a cell strainer) with 30× volume water. Next, the material was removed from the filter and incubated in 20× volume of 2:1 chloroform:methanol for 20 min at room temperature (e.g., about 20-22° C.). Next, the material was washed using a filter unit (e.g., a cell strainer) with 30× volume of water. The excess liquid was removed by blotting with a paper and the material was weighed thereafter. Next, the sample was submerged in 5 volumes of 0.5 M Acetic acid for 30 seconds. The sample was then incubated in 4× weight of 0.75% pepsin in 0.5M Acetic acid for 48 hours at room temperature (e.g., about 20-22° C.) while simultaneously mixing.

The sample was centrifuged through a 100 μm cell strainer by subjecting the sample to centrifugation at 500×g (Fisherbrand™ 100 μm Nylon mesh Cat #22362549). The sample was then centrifuged at 15,000×g and the clear supernatant on top was collected. The sample was then dialyzed against 100× volume of cold Tris Buffered Saline (TBS) at 4° C. for 24 hours (Spectrum Laboratories® 12-14 kDa dialysis membrane). The sample was subsequently dialyzed in 100× volume 8M urea at 4° C. for 16 hours. The sample was subsequently dialyzed in 100× volume 0.5% Chloroform in TBS at 4° C. for 16 hours. The sample was subsequently dialyzed in 1000× volume TBS at 4° C. for 16 hours. The sample was subsequently dialyzed in 1000× volume PBS at 4° C. for 16 hours. Lastly, the sample was aseptically collected from the dialysis bag under a tissue culture hood and stored at −20° C. until further use.

The product yield of the hAdipoGel was on average about 1 ml of hAdipoGel per initial gram of discarded human adipose tissue. The composition of hAdipoGel, as determined by mass spectrometry, was a mixture of defined size fragments from collagens COL1A1, COL1A2, COL3A1, COL4A2, COL5A2, and FBN1, achieved through specific chemical extraction and digestion protocols. Furthermore, as shown in FIG. 1, gel electrophoresis analysis of material extracted by guanidine hydrochloride (GuHCl) (labeled as “Extract”) and the final product (labeled as “hAdipoGel”) was performed. The final product, hAdipoGel, had many non-essential components removed. The hAdipoGel composition included COL1A1, COL1A2, COL3A1, COL4A2, COL5A2 fragments having an average size of about 20 kD. The size of the fragments in the hAdipoGel composition was identified to be in the following order: COL1A1>COL1A2>COL3A1>COL4A2>COL5A2 (i.e., COL1A1 had an average size that was greater than the rest of the fragments, and COL5A2 had an average size that was lesser than the rest of the fragments). The hAdipoGel composition was liquid at 5° C., but formed a three-dimensional hydrogel when warmed to 37° C.

### Example 2—Assessment of Proliferation and Differentiation of Mesenchymal Progenitor Cells Embedded in hAdipoGel Scaffolds

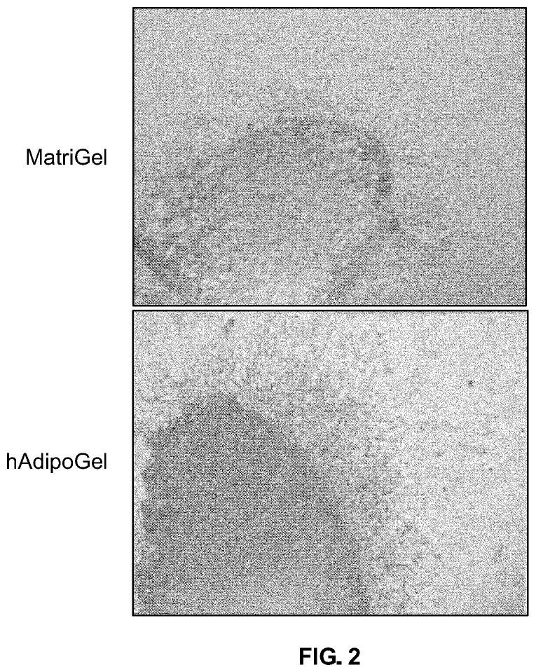

The proliferation and differentiation of mesenchymal progenitor/stem cell in hAdipoGel vs. Matrigel was examined. As shown in FIG. 2, explanted human adipose tissue was embedded in either Matrigel (top) or hAdipoGel (bottom). Mesenchymal progenitor cells sprouted from the tissue after 5 days in culture in either Matrigel or hAdipoGel. Thus, hAdipoGel was shown to support the embedding of tissue for three-dimensional culture in a similar manner as Matrigel.

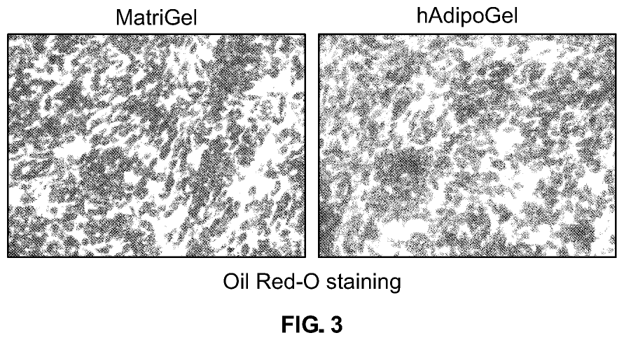

The mesenchymal progenitor cells that were produced from human explants in either Matrigel or hAdipoGel were plated and cultured with a differentiation medium to induce adipocyte differentiation. FIG. 3 shows microscopy images showing the accumulation of lipid as assessed by Oil Red-O staining. As can be seen in FIG. 3, the mesenchymal progenitor cells grown in both hAdipoGel and Matrigel underwent differentiation into adipocytes. There was no apparent difference between conditions; therefore, hAdipoGel was shown to support adipogenic differentiation as effectively as the Matrigel condition.

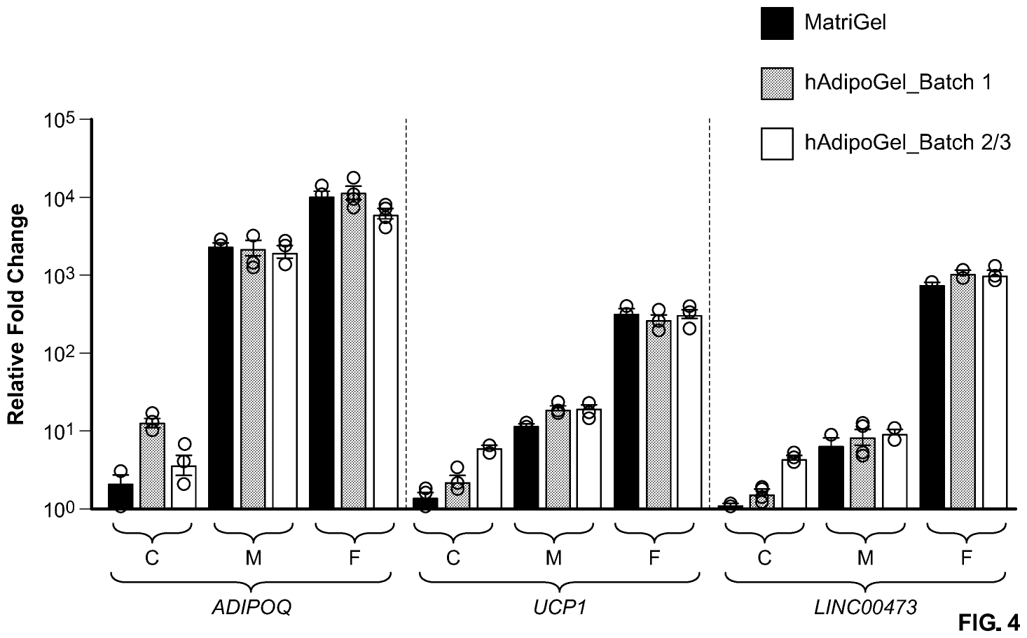

Next, marker expression of the differentiated adipocytes was characterized. As shown in FIG. 4, the expression of various markers in adipocytes generated from the mesenchymal progenitor cells grown in hAdipoGel or Matrigel were quantified. Mesenchymal progenitor cells grown in Matrigel (blue bars) or grown in two batches of hAdipoGel obtained from different donors (green and red bars) were plated and maintained in an undifferentiated state (C), induced to differentiate (M), or stimulated with forskolin after differentiation (F). The expression levels of genes are shown in the x-axis (Adiponectin, UCP1, or LINC00473). These results showed that the adipocytes generated from the mesenchymal progenitor cells grown in either hAdipoGel or Matrigel expressed similar markers.

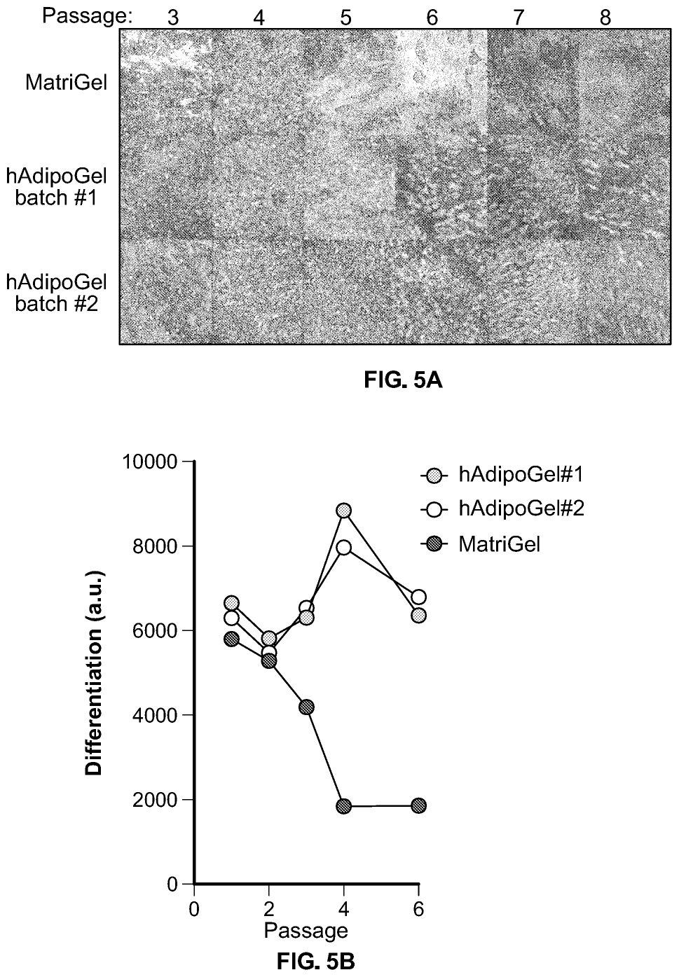

Next, the maintenance of multipotency of cells grown in the different types of hydrogels was tested. Mesenchymal progenitor cells were grown in Matrigel, and in two separate batches of hAdipoGel obtained from different donors. The mesenchymal progenitor cells were plated, allowed to reach confluence, and passaged at a 1:2 ratio for the number of passages indicated in FIG. 5A. At each passage, the capacity of cells to differentiate into adipocytes was assessed by Bodipy (green) staining. FIG. 5B shows a graph illustrating the quantification of Bodipy staining at each passage. As shown in FIGS. 5A and 5B, a greater number of cells is maintained over the number of passages in both of the hAdipoGel conditions. These results showed mesenchymal progenitor cells grown in hAdipoGel maintain multipotency over more passages compared to cells grown in Matrigel. Human mesenchymal progenitor cells proliferated in hAdipoGel at a similar rate to that seen in Matrigel. Importantly, the capacity of cells to retain multipotency was higher in hAdipoGel compared to Matrigel by a minimum of two passages. This means that the yield of human multipotent progenitor cells was at a minimum 8-times greater when grown in hAdipoGel compared to Matrigel, indicating lower genetic drift.

Next, a side-by-side comparison of cell implantation into immunocompromised mice was performed. In these experiments, human mesenchymal progenitor cells were primed in vitro to differentiate into adipocytes. The primed cells were resuspended in either Matrigel or hAdipoGel, and implanted subcutaneously into immunocompromised mice, which can tolerate human tissue. Blood from the mice was analyzed 4, 8, and 16 weeks later for the presence of circulating human hormones (e.g., adiponectin) produced by implanted cells. After 10 weeks, tissue formed from implanted cells was excised and analyzed by histochemistry.

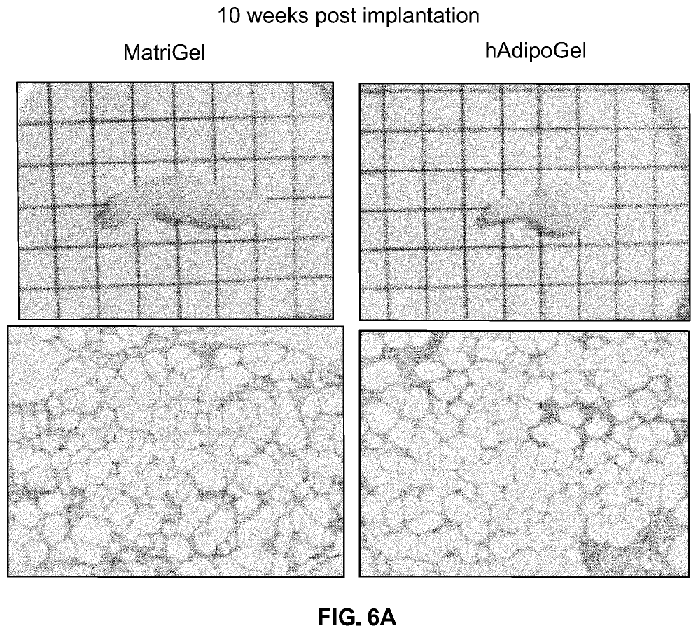

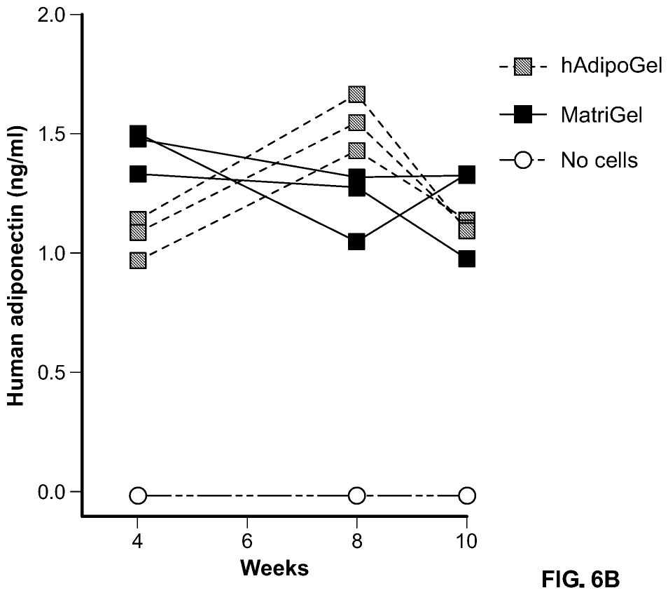

FIG. 6A shows photographs of the excised tissue (top row) and microscopy images of the histochemical staining of the excised tissue (bottom row). Images and histochemical analysis results were comparable between conditions. Serum from mice implanted with cell-loaded Matrigel (“Matrigel”), cell-loaded hAdipoGel (“hAdipoGel”), and control gel (“Matrigel No Cells”) was analyzed for human adiponectin content. FIG. 6B is a graph showing the adiponectin content in these three conditions (i.e., “Matrigel,” “hAdipoGel,” and “Matrigel No Cells”). Production of adiponectin from cells implanted in Matrigel or in hAdipoGel was statistically indistinguishable. Thus, these results showed mesenchymal progenitor cells resuspended in hAdipoGel or Matrigel implanted into nude mice form equally functional tissue. hAdipoGel supported development of well-vascularized, functional tissue, indistinguishable from that produced in Matrigel.

In summary, these data demonstrated hAdipoGel was superior to Matrigel in supporting proliferation if human mesenchymal progenitor/stem cells, and supporting multilineage differentiation (e.g., adipogenic differentiation).

### Example 3—Assessment of iPSC-Derived Human Beta Cells' Survival and Function when Embedded within an hAdipoGel Scaffolds

The purpose of this study was to determine whether iPSC-derived human beta cells would survive and respond to glucose when embedded within an hAdipoGel scaffold and implanted subcutaneously into mice. iPSC-derived human beta cells typically do not survive when implanted subcutaneously and have to be implanted under the kidney capsule.

C-peptide produced by the iPSC-derived human beta cells was measured in this study. C-peptide is released into the blood as a byproduct of the formation of insulin by the pancreas. In the pancreas, within beta cells, proinsulin, a biologically inactive molecule, is split apart to form one molecule of C-peptide and one molecule of insulin. Insulin is vital for the transport of glucose into the body's cells and is required on a daily basis. When insulin is released from the beta cells into the blood in response to increased levels of glucose, equal amounts of C-peptide are also released. Since C-peptide is produced at the same rate as insulin, it is useful as a marker of insulin production. Thus, C-peptide can be used as a measure of pancreatic beta cell function.

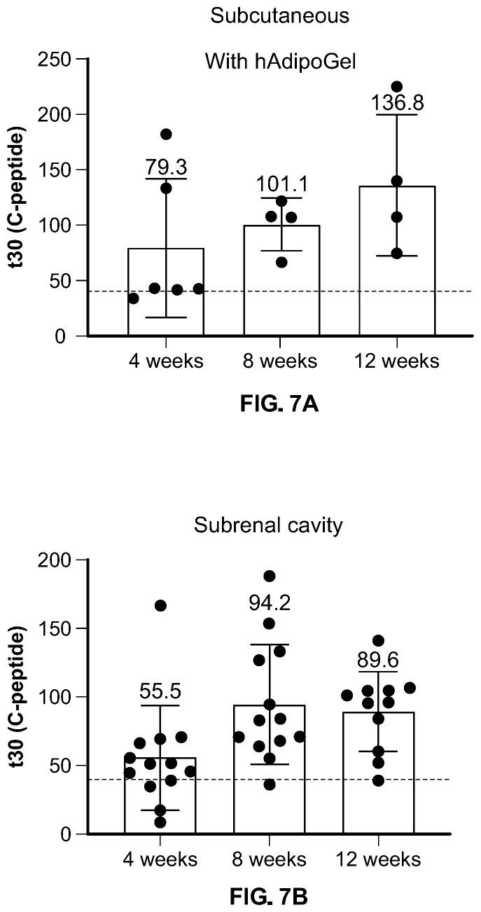

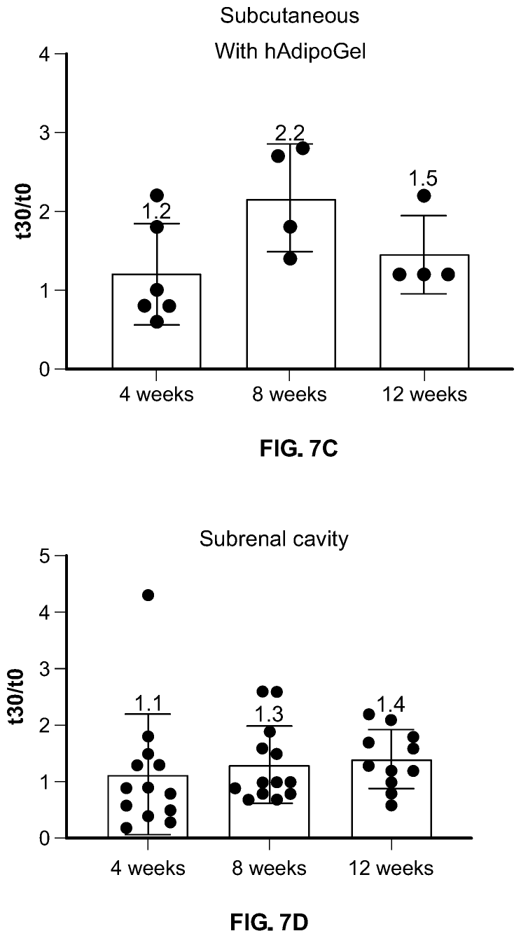

As shown in FIG. 7A, the iPSC-derived human beta cells embedded within an hAdipoGel scaffold and implanted subcutaneously survived for 12 weeks, as evidenced by their production of C-peptide. iPSC-derived human beta cells embedded within an hAdipoGel scaffold and implanted subcutaneously also produced higher levels of C-peptide than iPSC-derived beta cells implanted within a rodent subrenal cavity (e.g., a kidney capsule), as shown in FIG. 7B.

Next, to assess the function of the iPSC-derived human beta cells, the levels of C-peptide generated by the iPSC-derived human beta cells were measured before and after a glucose injection. As shown in FIG. 7C, the iPSC-derived beta cells encapsulated within the hAdipoGel scaffold and implanted subcutaneously in mice produced higher amounts of C-peptide in response to the glucose injection as compared to the iPSC-derived beta cells that were implanted within the rodent subrenal cavity.

Thus, this study demonstrated the ability of the hAdipoGel compositions to provide the appropriate environment to facilitate survival and viability of iPSC-derived beta cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-35. (canceled)

36. An isolated hydrogel composition, comprising one or more of collagen 1A1, collagen 1A2, collagen 3A1, collagen 4A2, collagen 5A2, and fibrillin-1, wherein the composition is derived from a mammalian adipose tissue, and wherein the composition is substantially free of nucleic acids and lipids.

37. The composition of claim 36, wherein the one or more of collagen 1A1, collagen 1A2, collagen 3A1, collagen 4A2, collagen 5A2, and fibrillin-1 are fragmented, and wherein a size of the one or more fragmented collagen 1A1, collagen 1A2, collagen 3A1, collagen 4A2, collagen 5A2, and fibrillin-1 ranges from about 10 kilodalton (kDa) to about 30 kDa.

38. The composition of claim 36, wherein the composition comprises one or more peptides of the one or more of collagen 1A1, collagen 1A2, collagen 3A1, collagen 4A2, collagen 5A2, and fibrillin-1, and wherein the composition is substantially free of cells and/or non-fibrous proteins.

39. The composition of claim 38, wherein collagen 1A1 is present at a concentration greater than a concentration of each of collagen 1A2, collagen 3A1, collagen 4A2, collagen 5A2, and fibrillin-1, and wherein fibrillin-1 is present at a concentration that is less than a concentration of each of collagen 1A1, collagen 1A2, collagen 3A1, collagen 4A2, and collagen 5A2.

40. The composition of claim 36, wherein collagen 1A1 is present at a concentration of about 20 weight (wt) %, collagen 1A2 is present at a concentration of about 18 wt %, collagen 3A1 is present at a concentration of about 17 wt %, collagen 4A2 is present at a concentration of about 16 wt %, collagen 5A2 is present at a concentration of about 15 wt %, and fibrillin-1 is present at a concentration of about 14 wt %.

41. The composition of claim 36, wherein collagen 1A1 is present at a concentration ranging from about 15 weight (wt) % to about 25 wt %, collagen 1A2 is present at a concentration ranging from about 13 wt % to about 23 wt % collagen 3A1 is present at a concentration ranging from about 12 wt % to about 22 wt % collagen 4A2 is present at a concentration ranging from about 11 wt % to about 21 wt % collagen 5A2 is present at a concentration ranging from about 10 wt % to about 20 wt % and fibrillin-1 is present at a concentration ranging from about 9 wt % to about 19 wt %.

42. The composition of claim 36, wherein the composition forms a gel when exposed to a temperature ranging from about 37 degrees Celsius to about 40 degrees Celsius, and wherein the composition is a liquid when exposed to a temperature ranging from about 1 degree Celsius to about 5 degrees Celsius.

43. A method of preparing an isolated hydrogel composition, the method comprising:
providing an adipose tissue sample from a subject;
freezing the adipose tissue sample;
slicing the adipose tissue sample into a sheet;
contacting the sheet with a denaturant to denature substantially all non-fibrous proteins in the adipose tissue sample;
mechanically processing the adipose tissue sample to lyse substantially all cellular material in the adipose tissue sample;
contacting the adipose tissue sample with a nuclease to remove substantially all nucleic acid material from the adipose tissue sample;
contacting the adipose tissue sample with an organic solvent to remove substantially all lipids from the adipose tissue sample;
contacting the adipose tissue sample with a protease to digest proteins in the adipose tissue sample; and
dialyzing the adipose tissue sample using a dialysis membrane.

44. The method of claim 43, wherein the method does not comprise contacting the adipose tissue sample with a detergent, wherein the denaturant is guanidine hydrochloride, wherein the nuclease is an endonuclease, and wherein the protease is pepsin.

45. The method of claim 43, wherein mechanically processing the adipose tissue sample comprises homogenizing the adipose tissue sample, and wherein the sheet has a thickness ranging from about 1 millimeter (mm) to about 3 mm.

46. The method of claim 43, wherein the organic solvent is a polar organic solvent and/or a non-polar organic solvent, and wherein the organic solvent is ethanol, methanol, chloroform, or any combination thereof.

47. The method of claim 43, wherein the protease comprises a protease-acid solution having a weight that is about four times the weight of the adipose tissue sample, wherein a concentration of the protease in the protease-acid solution is about 75%, and a concentration of the acid in the protease-acid solution is about 0.5 M, and wherein the dialysis membrane has a molecular weight cutoff ranging from about 12 kDa to about 14 kDa.

48. An isolated hydrogel composition prepared by the method of claim 43.

49. A method of culturing a cell or a population of cells, the method comprising suspending the cell or the population of cells in or contacting the cell or the population of cells with the isolated hydrogel composition of claim 36, under conditions sufficient for growth of the cell or the population of cells.

50. The method of claim 49, wherein the cell or the population of cells includes one or more of a stem cell, a progenitor cell, a pluripotent cell, an induced pluripotent stem cell (iPSC), and an iPSC-derived beta cell, wherein the isolated hydrogel composition promotes a growth of the cell or the population of cells, and wherein the cell or the population of cells include a cell that has been genetically altered.

51. The method of claim 49, wherein suspending the cell or the population of cells in the isolated hydrogel composition is performed at a temperature that is at least about 4 degrees Celsius or less, and wherein the isolated hydrogel composition is in a liquid or flowable state.

52. The method of claim 49, wherein contacting the cell or the population of cells with the isolated hydrogel composition is performed at a temperature ranging from about 6 degrees Celsius to about 40 degrees Celsius, and wherein the isolated hydrogel composition is in a solid, semi-solid, or gel state.

53. The method of claim 52, wherein contacting the cell or the population of cells with the isolated hydrogel composition is performed at a temperature of about 37 degrees Celsius.

54. A method of treating a lipodystrophy in a subject in need thereof, the method comprising:
obtaining a population of adipose progenitor cells from the subject;
optionally genetically altering one or more adipose progenitor cells from the population of adipose progenitor cells to correct an underlying genetic defect;
optionally differentiating the adipose progenitor cells into adipocytes;
suspending the adipose progenitor cells or the adipocytes in or contacting the adipose progenitor cells or the adipocytes with the isolated hydrogel composition of claim 36; and
implanting the isolated hydrogel composition comprising the adipose progenitor cells or the adipocytes in the subject.

55. A method of treating a subject in need thereof, the method comprising:
obtaining a population of progenitor cells, optionally from the subject;
optionally genetically altering the progenitor cells to correct an underlying genetic defect;
optionally differentiating the progenitor cells into differentiated cells;
suspending the progenitor cells or the differentiated cells in or contacting the progenitor cells or the differentiated cells with the isolated hydrogel composition of claim 36; and
implanting the isolated hydrogel composition comprising the progenitor cells or the differentiated cells in the subject.
